(19) United States
(12) Patent Application Publication
(10) Pub. No.: US 2025/0020606 A1
(43) Pub. Date: Jan. 16, 2025

(54) SYSTEM HAVING A SAMPLE STORE FOR PREPARING A SAMPLE MATERIAL IN THE CEMENT OR LIME INDUSTRY FOR CALORIMETRIC MEASUREMENT

(71) Applicants: thyssenkrupp Polysius GmbH, Beckum (DE); thyssenkrupp AG, Essen (DE)

(72) Inventors: Thomas RUETHER, Drensteinfurt (DE); Michael ENDERS, Muenster (DE)

(73) Assignees: thyssenkrupp Polysius GmbH, Beckum (DE); thyssenkrupp AG, Essen (DE)

(21) Appl. No.: 18/713,107

(22) PCT Filed: Nov. 22, 2022
(86) PCT No.: PCT/EP2022/082884

§ 371 Date: May 23, 2024

(30) Foreign Application Priority Data
Nov. 24, 2021 (BE) BE 2021/5915
Nov. 24, 2021 (DE) 10 2021 213 232.4

(51) Int. Cl. G01N 25/48

(52) U.S. Cl. CPC G01N 25/4826; G01N 25/486

(57) ABSTRACT

The present invention relates to a system 24 having a sample store 10 for preparation of a sample material in the cement or lime industry for a calorimetric measurement, having an accommodation body 12 having at least one or a multitude of storage sites 14 each for accommodating a sample vessel containing a sample material, wherein the sample store 10 has a temperature control device for cooling or heating the storage sites 14 of the accommodation body 12.

The invention relates to a system having a sample store for preparation of a sample material in the cement or lime industry for a calorimetric measurement, and to a method of determining the reactivity of a sample material.

In the production of clinker cement, it is customary to use spectroscopy and diffractometry analysis methods in order to predict the reactivity of clinker and cement. However, the use of alternative raw materials and fuels makes it difficult to make such a prediction, since minor ingredients that are not determined in the analysis also cause a change in reactivity of clinker and cement. Important examples of these are fluorine in the case of clinker mineralization, sulfur oxide from petcoke, or phosphorus when animal meal is used. In addition, it is also possible when chemistry and mineralogy are known for the dwell time of the raw materials in the furnace and process parameters such as furnace rotation and flame length to affect the reactivity. The understanding of such relationships is limited since there has to date been a lack of measurements close to the process in respect of reactivity of clinker and cement in many cases. It is possible to determine the reactivity of clinker and cement only with a considerable time delay analytically by compressive strength studies in mortar or in concrete. However, determination of compressive strength is too time-consuming to be able to use the results for process control.

DE 10 2014 018 489 A1 discloses a method of analysis of a cement material. A known means of closed-loop process control using reactivity data is that of isothermal heat flow calorimetry. This method utilizes the heat released from the sample over time as a measure of reactivity. It is thus possible using characteristic curves, in absolute terms and during the procedure, to measure and characterize the reaction characteristics and to use them as an input signal into the process controller.

US 2020/015 005 A1 discloses a sample mill.

In ascertaining the heat released from the sample over time, however, measurement errors regularly occur, which result in incorrect determination of the reactivity and corresponding incorrect process control.

Proceeding from this, it is an object of the present invention to provide an apparatus and a method of ascertaining isothermal heat flow calorimetry that has low proneness to error.

This object is achieved in accordance with the invention by a system for determining the reactivity of a sample material, having the features of independent apparatus claim 1, and by a method of determining the reactivity of a sample material, having the features of independent method claim 12. Advantageous developments will be apparent from the dependent claims.

The invention comprises a system for determining the reactivity of a sample material, having:
a metering device for metering the sample material into a sample vessel,
a sample store for adjusting the temperature of the sample material in the sample vessel, as described above,
a mixing apparatus for accommodating the sample vessel containing the sample material at controlled temperature and for mixing the sample material in the sample vessel, and
a calorimeter for ascertaining the reactivity of the mixed sample material.

The sample store, for preparation of a sample material in the cement or lime industry for calorimetric measurement, comprises:
an accommodation body having at least one or a multitude of storage sites each for accommodation of one sample vessel containing a sample material, and a temperature control device for cooling or heating the storage sites of the accommodation body.

The sample material is, for example, a clinker or a hydraulic binder having different compositions of various material components, for example clinker, sulfate carriers or grinding admixtures. Grinding admixtures are, for example, foundry sand, fly ash, pozzolan, limestone or calcined clay. The sample material has preferably been ground and is especially pulverulent.

A calorimetric measurement of the sample material is preferably used to ascertain the reactivity of the sample material. Preferably, for this purpose, the amount of heat emitted by the sample material per unit time is ascertained after addition of a trigger liquid. The calorimetric measurement is preferably isothermal.

The accommodation body is, for example, a solid metal block. The accommodation body is preferably in the shape of a box and has, for example, an internally hollow configuration. The accommodation body is optionally in one-piece form. The storage sites each serve to accommodate a sample vessel, where the storage sites are preferably designed such that a sample vessel is accommodated fully or partly within the storage site. Each storage site is preferably designed to accommodate exactly one sample vessel each. The sample vessel is, for example, a cylindrical ampoule which is preferably closable with a lid, especially by means of a screw closure. The storage sites are, for example, as essentially vertical cutouts, especially drillholes from the top into the accommodation body. The storage sites are cylindrical, for example, and have a round, especially circular, constant cross section. The depth of the storage sites preferably corresponds to at least 5% of the height of the sample vessel to be accommodated therein, preferably not more than 4 times the height of the sample vessel, especially 0.9-2 times the height of the sample vessel, preferably in particular 0.5 times the height of the sample vessel. The storage sites are preferably arranged alongside one another in a horizontal plane, especially in rows, and preferably spaced apart uniformly from one another. In particular, the number of storage sites is 10 to 100, preferably 20 to 80, in particular 50. It is likewise conceivable that a multitude of storage sites are in a stacked arrangement alongside one another in a vertical direction within the accommodation body.

The temperature control device serves the temperature control, especially cooling or heating, of the storage sites, preferably of the sample material in the sample vessels that are stored in the storage sites. The temperature of the sample vessel, after a particular dwell time of the sample vessel in the respective storage site, preferably corresponds to the temperature of the storage site. This achieves optimal temperature control of the sample vessel before the reactivity of the sample material is ascertained and hence increases the measurement accuracy.

The sample store is preferably part of a system for determining the reactivity of a sample material from the cement or lime industry. For example, identical products are sampled and compared at different sampling times. One finding by the inventors is that it is necessary for a reliable determination of reactivity, for example via the initial peak of the heat emitted, that there is a negligibly small temperature differential between the sample material and the internal temperature of the measurement space of the calorimeter at the start of the measurement. It is obvious that a temperature of the sample material different from the internal calorimeter temperature will lead to an incorrect determination of the reactivity of the sample material since this additional amount of heat resulting from the difference in temperature between the sample material and the interior of the isothermal heat flow calorimeter will also be added to the enthalpy of reaction of the sample. The inventors have found that, especially for the measurement of rapid isothermal heat flow measurements close to the process, differences in temperature between the sample material and the internal temperature of the heat flow calorimeter are detrimental to the interpretation of the measurement result.

One finding by the inventors is that, as well as the actual ambient temperature, different temperatures of process samples in particular can have an unfavorable heat signature for heat flow calorimetry. Examples of these are samples of clinker shortly after production in the clinker furnace or else cement samples that still have a temperature above ambient temperature from the grinding process or else insufficiently exact setting of the ambient temperature around the generally automated isothermal heat flow calorimeter. The inventors have therefore found that the sample material provided for isothermal heat flow calorimetry ideally has to be adjusted thermally to the internal temperature of the heat flow calorimeter before commencement of the measurement.

Reliable determination of reactivity offers the benefit of an optimal process regime, with direct comparison of products sampled at different sampling times from cement grinding and the ability to implement a closed-loop control intervention, for example via a change in product fineness or in clinker content. Analogously, the clinker can be controlled to a target value via variation in the raw materials and fuels within the scope of a determination of reactivity.

In a first embodiment, the temperature control device has a heat transfer medium circuit with a heating/cooling device. In particular, the temperature control device comprises at least one or a multitude of conduits for conducting a heat transfer medium, where the conduits preferably extend at least partly through the accommodation body. The heat transfer medium is, for example, a gas, such as air, or a liquid heat transfer medium, for example water, thermal oil, glycol or salt solutions or mineral oil. The conduit extends, for example, beneath the storage sites, especially in a plane, preferably in spiral or helical form. The conduit is preferably arranged at least partly around the storage sites.

The heating/cooling device is preferably an aggregate designed for heating and/or cooling of a the heat transfer medium. For example, a heating/cooling device comprises a heat exchanger or an electrical heating or cooling unit. The temperature control device especially comprises a buffer storage medium in order to buffer short-term fluctuations in temperature of the heat transfer medium. In addition, the temperature control device has, for example, a fan or a compressor and in particular further pipe conduits for connection of the components of the temperature control device.

In a further embodiment, the accommodation body is formed from a metal. The metal is preferably aluminum or steel. The storage sites formed in the accommodation body preferably have a geometry corresponding roughly to the geometry of a sample vessel, such that it can be accommodated in the storage site, preferably with only a very small amount of play, if any. An accommodation body in metal form ensures optimal thermal conductivity, such that heat or cooling introduced into the sample vessel via the conduits of the heat transfer medium is passed onward in an optimal manner to the storage sites and the sample vessels stored therein.

In a further embodiment, the sample store has at least one or a multitude of conduits for conducting an activation liquid, such that the temperature of the activation liquid in the conduits is adjustable by means of the temperature control device. The conduits for conducting the activation liquid preferably extend through the accommodation body. In particular, the conduits for conducting the activation liquid are arranged in the accommodation body such that their temperature is controlled, and they are in particular cooled or heated, by the conduits for conducting the heat transfer medium. The conduits for conducting the activation liquid are preferably arranged directly alongside the conduits for conducting the heat transfer medium. It is likewise conceivable that the conduits for conducting the activation liquid extend completely or partly along the outside of the accommodation body. The activation liquid is, for example, water or distilled water, which is usable for activation of the sample material. For example, the conduits for conducting the activation liquid are connected to a reservoir, especially a tank, for intermediate storage of activation liquid. The reservoir is disposed, for example, within the sample store, and its temperature is controllable by means of the heat transfer medium conducted within the conduits. It is likewise conceivable that the reservoir is disposed outside the sample store and is connected thereto via conduits, especially by the conduits for conducting the activation liquid. The dwell time of the activation liquid within the sample store, especially within the conduits for conducting the activation liquid that run through or along the accommodation body, is preferably adjustable.

In a further embodiment, the accommodation body has an upper region and a separate lower region, wherein the conduits for conducting the heat transfer medium and/or the conduits for conducting the activation liquid are disposed in the lower region. The lower region and the upper region are preferably releasably connected to one another, for example by screw connection.

The conduits for conducting the activation liquid and the conduits for conducting the heat transfer medium are preferably disposed exclusively in the lower region. The lower region is preferably in the shape of a box, such that the conduits are reachable from above into the lower region, for example for maintenance. The storage sites are preferably disposed exclusively in the upper region of the accommodation body. A divided accommodation body simplifies the maintenance of the conduit for conducting the heat transfer medium and the activation liquid.

In a further embodiment, the sample store has at least one temperature measurement device for ascertaining the temperature of the accommodation body and/or the storage sites. There are preferably a multitude of temperature measurement devices mounted on the accommodation body and, for example, spaced apart uniformly from one another. In particular, the temperature measurement device is designed such that it ascertains a temperature profile preferably over at least one measurement plane within the accommodation body.

In a further embodiment, the sample store has an open-loop/closed-loop control device designed to control the temperature of the sample store by open-loop/closed-loop control depending on the temperature ascertained by the temperature measurement device. Open-loop/closed-loop control is preferably understood to mean open-loop and/or closed-loop control. The temperature measurement device is especially connected to the open-loop/closed-loop control device for transmission of the temperature ascertained.

The temperature of the sample store, especially of the accommodation body, preferably of the storage sites, is preferably adjusted by means of the open-loop/closed-loop control device. A target temperature value is preferably recorded in the open-loop/closed-loop control device. The open-loop/closed-loop control device is preferably designed such that it compares the ascertained temperature of the sample store, especially of the storage sites, with the target temperature value and, in the event of variance of the temperature ascertained from the target temperature value, increases or reduces the temperature of the sample store, especially of the storage sites, such that this corresponds to the target temperature value.

The open-loop/closed-loop control device is preferably connected to the temperature control device for adjusting the temperature of the sample store. In order to alter the temperature of the sample store, especially the storage sites, the temperature of the heat transfer medium flowing through the conduit is adjusted, especially increased or reduced. The heat transfer medium is preferably heated or cooled by means of a heating or cooling device, such as a heat exchanger or an electrical cooling or heating aggregate. The target temperature value of the sample store corresponds, for example, to a temperature value ascertained by a calorimeter disposed downstream of the sample store and separately therefrom, especially an isothermal calorimeter, and especially the temperature in a measurement space of the calorimeter.

In a further embodiment, the sample store has an open-loop/closed-loop control device designed to control the dwell time of the sample vessel in the storage sites by open-loop/closed-loop control depending on the temperature ascertained. The open-loop/closed-loop control device is preferably designed such that it calculates the temperature differential between the sample store, especially the storage sites, and the target temperature value or the temperature ascertained in the calorimeter, and controls the dwell time of the sample vessel in the sample store by open-loop/closed-loop control depending on the calculated temperature differential. The dwell time ascertained, especially together with the material indices such as fineness and mass, preferably corresponds to a dwell time of the sample vessel within the sample store. The dwell time is preferably chosen such that the temperature of the sample vessel and, for example, the contents thereof are matched to the temperature of the accommodation body, especially storage sites, via the dwell time.

In a further embodiment, the storage sites are formed as cutouts in the accommodation body, such that the sample vessels can be arranged at least partly within the accommodation body. The cutouts are, for example, drillholes or depressions in the accommodation body and are especially spaced apart uniformly from one another and preferably arranged in several rows. The cutouts are preferably such that the sample vessel is fixable in a cutout. The depth of the cutouts preferably corresponds to at least 5% of the height of a sample vessel, especially 4 times the height of a sample vessel, preferably twice the height of a sample vessel, especially 0.5 to 0.9 times the height of the sample vessel.

The metering device, the sample store, the mixing apparatus and the calorimeter are preferably separate components. In particular, the material sample in the sample vessel is stored in the sample store and transferred from the sample store into the mixing apparatus and from the mixing apparatus into the calorimeter in a manual or automated manner.

The metering device comprises, for example, a balance for ascertaining the weight of the sample material. The metering device is preferably operable manually or automatically. The metering device preferably comprises a conveying element for conveying the sample material, for example pneumatically or mechanically.

The mixing apparatus preferably serves to prepare the sample material for a calorimetric measurement and has, for example, an accommodation device for accommodating the sample vessel. The accommodation device is, for example, a capsule, a mixing chamber or a clamp device by means of which the sample vessel filled with the sample material is fixable. The mixing apparatus additionally has a frame. There are preferably vibrators mounted on the accommodation device of the mixing apparatus, which are preferably driven pneumatically and can set the sample vessel in a vertical and/or horizontal vibration. The accommodation device of the mixing apparatus is especially connected to the frame via vibration dampers that serve to secure the accommodation device to the frame, such that the vibration of the accommodation device is barely transmitted to the frame, if at all.

A calorimeter, especially an isothermal heat flow calorimeter, is designed such that it ascertains the heat of reaction released by the sample material. The heat of reaction released and the progression of the release of heat over time are characteristic of the reactivity of a sample material, especially a binder. The addition of an activation liquid, for example water, to the material components starts the hydration process, with release of the energy stored in the material components in the form of heat of reaction. The calorimetric measurement device enables simple and rapid ascertainment of the reactivity of the sample material.

In a further embodiment, a further metering device is provided for metering of an activation liquid into the sample vessel accommodated in the mixing apparatus. The activation liquid is preferably metered into the sample vessel before it is mixed in the mixing apparatus. For metering of the activation liquid, the sample vessel, for example, is accommodated in the mixing apparatus and especially open, such that the metering device can meter the activation liquid into the sample vessel. The temperature of the activation liquid is preferably adjusted prior to the metering into the sample vessel. In particular, the further metering device is designed to adjust the temperature of the activation device.

In a further embodiment, the calorimeter has a temperature measurement device for ascertaining the temperature of the calorimeter, and wherein the temperature measurement device is connected to the open-loop/closed-loop control device for transmission of the temperature ascertained. The calorimeter preferably has a measurement space in which the calorimetric measurement is effected. The temperature measurement device is preferably disposed in the measurement space. The temperature value ascertained is preferably a target temperature value for adjusting the temperature of the sample store, especially the storage sites.

For open-loop/closed-loop control of the temperature of the calorimeter, especially of the measurement space of the calorimeter, the open-loop/closed-loop control device is preferably designed such that it compares the ascertained temperature data from the calorimeter, especially from the measurement space of the calorimeter, with a predetermined target temperature value and, in the event of variance of the temperature ascertained from the target temperature value, increases or reduces the temperature of the calorimeter, especially of the measurement space of the calorimeter, such that it corresponds to the target temperature value.

The invention also encompasses a method of ascertaining the reactivity of a sample material, comprising the following steps:
metering the sample material into a sample vessel,
adjusting the temperature of the sample vessel in a sample store, as described above,
for example adjusting the temperature of an activation liquid,
feeding the sample vessel into a mixing apparatus for mixing the sample material in the sample vessel, and
ascertaining the reactivity of the sample material in the vessel.

The details and benefits described in relation to the system for determining the reactivity of a sample material are likewise applicable in a corresponding manner for the purposes of a method to the method of determining the reactivity of a sample material.

In a further embodiment, the temperature within the calorimeter and/or the temperature of the accommodation body is ascertained, where the temperature of the accommodation body of the sample store is controlled by open-loop/closed-loop control depending on the temperature ascertained.

In a further embodiment, the dwell time of the sample vessel in the sample store is preferably controlled by open-loop/closed-loop control depending on the temperature ascertained prior to commencement of the calorimetric measurement.

In a further embodiment, the temperature of an activation liquid in the sample store is controlled, followed by metering of the activation liquid into the sample vessel. The dwell time of the activation liquid in the sample store is preferably controlled by open-loop/closed-loop control depending on the temperature ascertained prior to commencement of the calorimetric measurement.

### DESCRIPTION OF THE DRAWINGS

The invention is elucidated in detail hereinafter by multiple working examples with reference to the appended figures.

FIG. 1 shows a schematic diagram of a sample store in a perspective view in one working example.

FIG. 2 shows a schematic diagram of a sample store in a top view of the lower region of the accommodation body in a further working example.

FIG. 3 shows a schematic diagram of a sample store in a cross-sectional view in a further working example.

FIG. 4 shows a schematic diagram of a system for determining the reactivity of a sample material in a further working example.

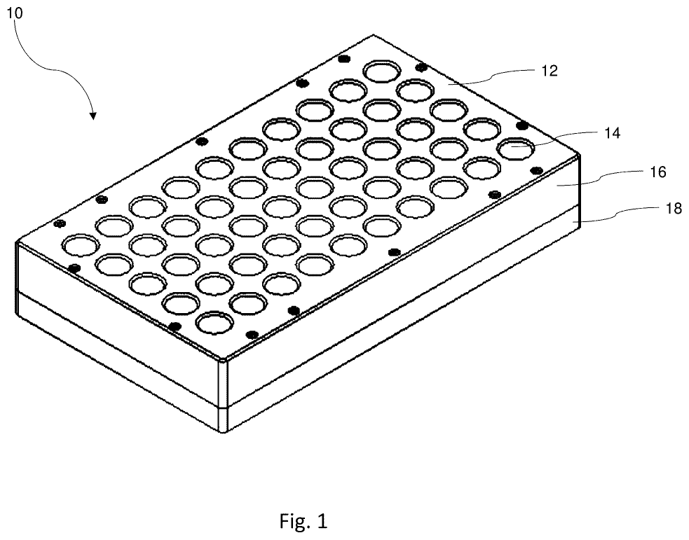

FIG. 1 shows a sample store 10 for accommodation of a multitude of sample vessels (not shown in FIG. 1) in which sample material is stored. The sample material is, for example, a clinker or a hydraulic binder having different compositions of different material components, for example clinker, sulfate carrier or grinding admixtures. Grinding admixtures are, for example, foundry sand, fly ash, pozzolan, limestone or calcined clay. The sample material has preferably been ground and is in particular in pulverulent form.

The sample store 10 has an accommodation body 12 having a multitude of accommodation means, especially storage sites 14. Each storage site 14 is preferably designed to accommodate exactly one sample vessel each. The sample vessel is, for example, an ampoule which is preferably closable with a lid. The lid can be screwed onto the ampoule, for example, by a thread in the form of a screw closure. The sample vessel preferably has a round, especially circular, cross section. The sample vessel is preferably formed from HD-PE (high density polyethylene), PET, polycarbonate, polypropylene or polystyrene.

The accommodation body 12 is preferably formed from a metal, for example aluminum or steel. In particular, the accommodation body 12 is in the form of a box. By way of example, the accommodation body 12 in FIG. 1 is in two-part form and has an upper region 16 and a lower region 18. It is likewise conceivable that the accommodation body 12 is in one-part or multipart form with more than two parts.

The upper region 16 of the accommodation body 12 is, for example, in the form of a box or of a solid block and has the storage sites 14. The storage sites 14 are formed, for example, as essentially vertical cutouts, especially drillholes from the top into the accommodation body 12, preferably the upper region 16 of the accommodation body 12. The storage sites 14 are cylindrical, for example, and have a round, especially circular, constant cross section. The depth of the storage sites 14 preferably corresponds to at least 5% of the height of the sample vessel to be accommodated therein, preferably not more than four times the height of the sample vessel, in particular 0.9-2 and in particular 0.5 times the height of the sample vessel.

By way of example, the sample store 10 has fifty storage sites that are arranged alongside one another in rows and are preferably spaced apart uniformly from one another. In particular, the number of storage sites is ten to one hundred, preferably twenty to eighty, especially fifty.

The lower region 18 of the accommodation body 12 is, for example, in the form of a box or of a solid block and preferably has at least one conduit, especially a multitude of conduits for conducting a heat transfer medium and/or an activation liquid. The heat transfer medium is, for example, a gas, such as air, or a liquid heat transfer medium, for example water, thermal oil, glycol or salt solutions or mineral oil. The upper region 16 and the lower region 18 of the accommodation body 12 are preferably connected to one another via a connecting means, such as screws, or, for example, cohesively bonded to one another, for example welded. In particular, the upper region 16 and the lower region 18 of the accommodation body 12 are releasably connected to one another.

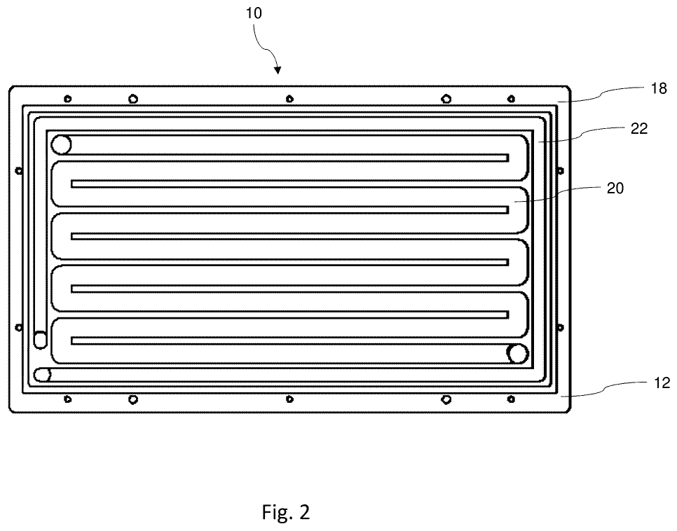

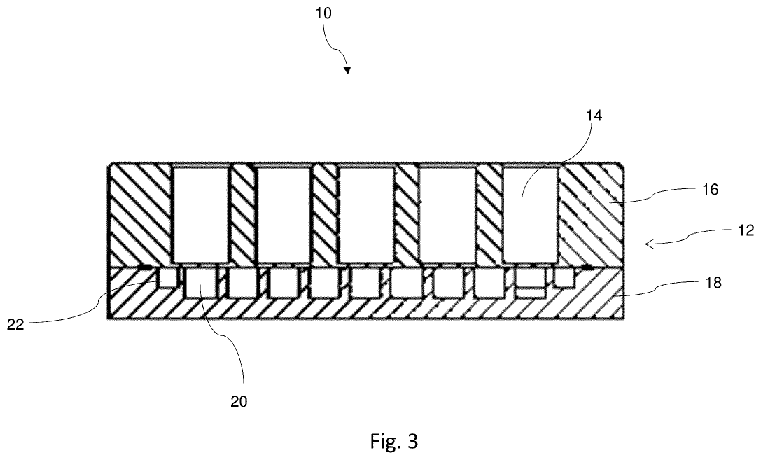

FIG. 2 shows a view of a sample store 10 top view of the lower region of the accommodation body 12, and FIG. 3 shows a cross-sectional view of the sample store. The sample store 10 has, by way of example, a conduit 20 for conducting a heat transfer medium. The heat transfer medium is, for example, a gas, such as air, or a liquid heat transfer medium, for example water, thermal oil, glycol or salt solutions or mineral oil. The conduit 20 is preferably part of a temperature control device which, in addition to the conduit 20, comprises a heat transfer medium circuit (not shown). The temperature control device preferably has a heat exchanger or heating/cooling aggregate to control the temperature of the heat transfer medium. In addition, the temperature control device has a fan or a compressor and, in particular, further pipe conduits for connection of the components.

The conduit 20 is preferably disposed beneath the storage sites 14. It is likewise conceivable that the conduit 20 at least partly surrounds the storage sites 14, especially in the lower regions thereof. The conduit 20 is preferably helical. In particular, the conduit 20 is arranged in a plane and extends across the plane in such a way, for example in spiral form or via a multitude of windings.

The sample store 10 has, by way of example, a further conduit 22 for conducting an activation liquid, for example water or distilled water. The conduit 22 for conducting an activation liquid is especially arranged around the outside of the conduit 20 for conducting the heat transfer medium and extends by way of example within the lower region 16 of the accommodation body 12 from the inside along the lateral faces of the lower region 16 of the accommodation body 12. The conduit 22 for conducting an activation liquid is, for example, part of a circuit (not shown) for the activation liquid, where the circuit preferably comprises a tank for storing an activation liquid and further conduits for connecting the tank to the sample store 10. The conduit 22 for conducting an activation liquid has, by way of example, a smaller diameter than the conduit 20 for conducting the heat transfer medium.

The conduit 20 for conducting the heat transfer medium serves to control the temperature of the sample vessel in the respective storage sites 14. The temperature of the heat transfer medium is preferably adjustable. The conduit 22 for conducting an activation liquid serves to control the temperature of the activation liquid, such that it preferably has the same temperature as the sample vessel, especially the sample material within the sample vessel.

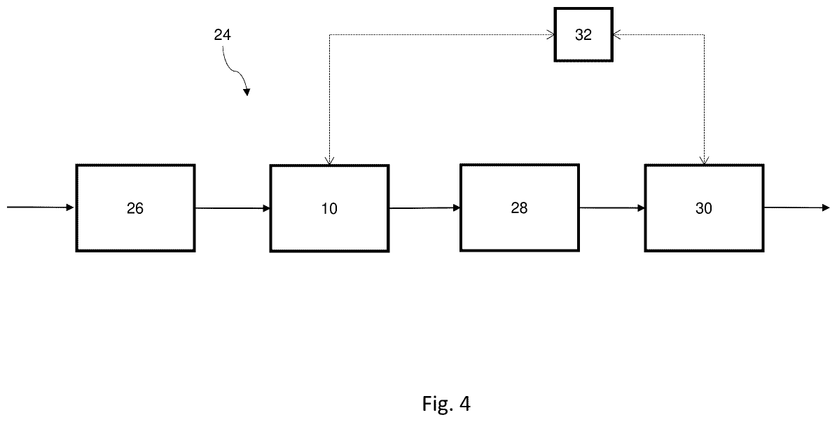

FIG. 4 shows a schematic diagram of a system for determining the reactivity of a sample material. The system 24 comprises a metering device 26 for metering sample material into a sample vessel. The metering device 26 comprises, for example, a balance for determining the sample weight and for metered addition of a predetermined weight of sample material into the sample vessel. The system 24 further comprises a sample store as described above with reference to FIGS. 1 to 3. The sample vessel filled with the sample material in the metering device 26 is fed manually or automatically into the sample store 10 and preferably accommodated in a storage site 14 in the sample store 10.

The system 24 preferably further comprises a mixing apparatus 28 for mixing of the sample material in the sample vessel. The mixing apparatus 28 preferably has a further metering device for metering of an activation liquid into the sample vessel. For this purpose, the sample vessel is preferably opened, especially screwed open, and the activation liquid is injected into the sample vessel. Subsequently, the sample vessel is preferably closed again, and the sample material is mixed with the activation liquid in the mixing apparatus 28.

The mixing apparatus 28 preferably serves for preparation of the sample material for a calorimetric measurement, especially for production of paste from a sample and an activation liquid. A calorimetric measurement is especially an ascertainment of the heat released by the sample material after addition of a trigger liquid, for example water or distilled water, to the sample material. The heat released is a measure of the energy stored in the sample material and the release of this energy over time.

The mixing apparatus 28 has, for example, an accommodation device for accommodation of the sample vessel. The accommodation device is, for example, a capsule, a mixing chamber or a clamp device by means of which the sample vessel filled with the sample material is fixable. The mixing apparatus 28 additionally has a frame. There are preferably vibrators mounted on the accommodation device of the mixing apparatus, which are preferably driven pneumatically and can set the sample vessel in a vertical and/or horizontal vibration. The accommodation device of the mixing apparatus 28 is especially connected to the frame via vibration dampers that serve to secure the accommodation device on the frame, such that the vibration of the accommodation device is barely transmitted to the frame, if at all.

The sample vessels stored intermediately in the sample store 10, the temperatures of which have been adjusted therein, are sent to the mixing apparatus 28. The sample vessels are preferably sent to the mixing apparatus 28 only when they have a particular predetermined target temperature.

The system 24 additionally includes a calorimeter 30, especially an isothermal heat flow calorimeter, which serves to determine the reactivity of the sample material. The sample vessels mixed in the mixing apparatus 28 are preferably sent individually, or several sample vessels simultaneously, to individual measurement spaces in the calorimeter.

By way of example, the system 24 additionally has an open-loop/closed-loop control device 32. It is likewise conceivable that the open-loop/closed-loop control device 32 is part of the sample store 10. The open-loop/closed-loop control device 32 is preferably connected to the calorimeter 30 and the sample store 10 in such a way that it controls the temperature of the sample store 10 and/or the temperature of the calorimeter 30 by open-loop/closed-loop control. The temperature of the storage sites 14 and/or the temperature of a measurement space of the calorimeter 30 are preferably adjusted by means of the open-loop/closed-loop control device 32. The sample store 10 and/or the calorimeter 30 preferably each have a temperature measurement device. The temperature measurement device mounted in the sample store 10 is especially designed and arranged so as to ascertain the temperature of at least one or more than one storage site 14. The temperature measurement device disposed in the calorimeter 30 is preferably designed and arranged so as to ascertain the temperature of the measurement space of the calorimeter. The temperature measurement devices are especially connected to the open-loop/closed-loop control device 32 for transmission of the temperature data ascertained.

There is preferably a target temperature value recorded in the open-loop/closed-loop control device 32. The open-loop/closed-loop control device 32 is preferably designed such that it compares the ascertained temperature data from the sample store 10, especially the storage sites 14, with the target temperature value and, in the event of variance of the temperature ascertained from the target temperature value, increases or reduces the temperature of the sample store 10, especially the storage sites 14, such that this corresponds to the target temperature value.

The open-loop/closed-loop control device 32 is preferably connected to the temperature control device for adjusting the temperature of the sample store 10. In order to alter the temperature of the sample store 10, especially the storage sites 14, the temperature of the heat transfer medium flowing through the conduit 20 is adjusted, especially increased or reduced. The heat transfer medium is preferably heated or cooled by means of a heating or cooling device, such as a heat exchanger or an electrical cooling or heating aggregate. The target temperature value of the sample store 10 corresponds, for example, to the ascertained temperature value of the calorimeter 30, especially the temperature in the measurement space of the calorimeter 30.

For open-loop/closed-loop control of the temperature of the calorimeter 30, especially the measurement space of the calorimeter 30, the open-loop/closed-loop control device 32 is preferably designed such that it compares the ascertained temperature data from the calorimeter 30, especially the measurement space of the calorimeter 30, with the target temperature value and, in the event of variance of the temperature ascertained from the target temperature value, increases or reduces the temperature of the calorimeter 30, especially the measurement space of the calorimeter 30, such that it corresponds to the target temperature value.

The open-loop/closed-loop control device 32 is preferably designed such that it controls the dwell time of the sample vessel in the sample store by open-loop/closed-loop control depending on the ascertained temperature of the sample store 10, especially the storage sites 14. The open-loop/closed-loop control device 32 is preferably designed such that it calculates the temperature differential between the sample store 10, especially the storage sites 14, and the target temperature value or the temperature ascertained in the calorimeter 30 and controls the dwell time of the sample vessel in the sample store 10 by open-loop or closed-loop control depending on the calculated temperature differential.

### LIST OF REFERENCE NUMERALS

10 sample store
12 accommodation body
14 storage sites
16 upper region of the accommodation body
18 lower region of the accommodation body
20 conduit for conducting a heat transfer medium
22 conduit for conducting an activation liquid
24 system for determining the reactivity of a sample material
26 metering device
28 mixing apparatus
30 calorimeter
32 open-loop/closed-loop control device

1-15. (canceled)

16. A system for determining the reactivity of a sample material, comprising:
a metering device for metering the sample material into a sample vessel,
a sample store for adjusting the temperature of the sample material in the sample vessel,
a mixing apparatus for accommodating the sample vessel containing the sample material at controlled temperature and for mixing the sample material in the sample vessel, and
a calorimeter for ascertaining the reactivity of the mixed sample material,
wherein the sample store, for preparation of a sample material in the cement or lime industry for calorimetric measurement, includes an accommodation body having at least one or a multitude of storage sites each for accommodation of one sample vessel containing a sample material, wherein the sample store has a temperature control device for cooling or heating the storage sites of the accommodation body.

17. The system as claimed in claim 16, wherein the temperature control device comprises conduits for conducting the heat transfer medium and a heating/cooling device.

18. The system as claimed in claim 17, wherein the accommodation body has an upper region and a separate lower region and wherein the conduits for conducting the heat transfer medium are disposed in the lower region.

19. The system as claimed in claim 16, wherein the accommodation body is formed from a metal.

20. The system as claimed in claim 16, wherein the sample store has conduits for conducting an activation liquid, such that the temperature of the activation liquid in the conduits is adjustable by the temperature control device.

21. The system as claimed in claim 20, wherein the accommodation body has an upper region and a separate lower region and wherein the conduits for conducting the activation liquid are disposed in the lower region.

22. The system as claimed in claim 16, wherein the sample store has at least one temperature measurement device for ascertaining the temperature of the accommodation body and/or the storage sites.

23. The system as claimed in claim 22, wherein the sample store has an open-loop/closed-loop control device designed to control the temperature of the sample store by open-loop/closed-loop control depending on the temperature ascertained with the temperature measurement device.

24. The system as claimed in claim 22, wherein the sample store has an open-loop/closed-loop control device designed to control the dwell time of the sample vessel in the storage sites by open-loop/closed-loop control depending on the temperature ascertained with the temperature measurement device.

25. The system as claimed in claim 16, wherein the storage sites take the form of recesses in the accommodation body, such that the sample vessels can be arranged at least partly within the accommodation body.

26. The system as claimed in claim 16, wherein a further metering device is provided for metering an activation liquid into the sample vessel accommodated in the mixing apparatus.

27. The system as claimed in claim 16, wherein the calorimeter has a temperature measurement device for ascertaining the temperature of the calorimeter and wherein the temperature measurement device is connected to the open-loop/closed-loop control device for transmission of the temperature ascertained.

28. A method of ascertaining the reactivity of a sample material with a system comprising a metering device for metering the sample material into a sample vessel, a sample store for adjusting the temperature of the sample material in the sample vessel, a mixing apparatus for accommodating the sample vessel containing the sample material at controlled temperature and for mixing the sample material in the sample vessel, and a calorimeter for ascertaining the reactivity of the mixed sample material, wherein the sample store, for preparation of a sample material in the cement or lime industry for calorimetric measurement, includes an accommodation body having at least one or a multitude of storage sites each for accommodation of one sample vessel containing a sample material, and wherein the sample store has a temperature control device for cooling or heating the storage sites of the accommodation body, the method comprising:
metering the sample material into a sample vessel,
adjusting the temperature of the sample vessel in a sample store,
feeding the sample vessel into a mixing apparatus for mixing the sample material in the sample vessel, and
ascertaining the reactivity of the sample material in the sample vessel by a calorimeter.

29. The method as claimed in claim 28, wherein the temperature within the calorimeter, and/or the temperature of the accommodation body is ascertained and wherein the temperature of the accommodation body of the sample store is controlled by open-loop/closed-loop control depending on the temperature ascertained.

30. The method as claimed in claim 28, wherein the dwell time of the sample vessel in the sample store is controlled by open-loop/closed-loop control depending on the temperature ascertained.

31. The method as claimed in claim 28, wherein the temperature of a trigger liquid is adjusted in the sample store and wherein the trigger liquid is then metered into the sample vessel.
